(19) United States
(12) Patent Application Publication
(10) Pub. No.: US 2025/0121096 A1
(43) Pub. Date: Apr. 17, 2025

(54) ENGINEERED LIVER-SPECIFIC ENHANCERS AND THEIR APPLICATIONS

(71) Applicant: Sichuan Real&Best Biotech Co., Ltd., Chengdu (CN)

(72) Inventors: Biao Dong, Chengdu (CN); Li Yang, Chengdu (CN)

(73) Assignee: Sichuan Real&Best Biotech Co., Ltd., Chengdu (CN)

(21) Appl. No.: 18/919,331

(22) Filed: Oct. 17, 2024

Related U.S. Application Data
(63) Continuation of application No. PCT/CN2024/092202, filed on May 10, 2024.
(60) Provisional application No. 63/510,993, filed on Jun. 29, 2023.

(30) Foreign Application Priority Data
May 11, 2023 (WO) PCT/CN2023/093398

(51) Int. Cl. A61K 48/00; A61K 38/37; A61P 7/04; C07K 14/755; C12N 15/86

(52) U.S. Cl. CPC A61K 48/0058; A61K 38/37; A61P 7/04; C07K 14/755; C12N 15/86; C12N 2750/14143; C12N 2830/008

(57) ABSTRACT

The present invention relates to engineered liver-specific enhancers, synthetic promoters containing the enhancers, expression vectors containing the synthetic promoters, as well as methods of using the enhancer or the expression vector thereof to address the need in the field, such as treatment of various genetic diseases or conditions associated with the liver. In some embodiments, the engineered enhancer comprises one or more DNA binding sites for transcription factors.

### CROSS-REFERENCE TO RELATED APPLICATIONS

The present application is a Continuation Application of PCT Application No. PCT/CN2024/092202, filed on May 10, 2024, which claims priority to PCT Application No. PCT/CN2023/093398, filed on May 11, 2023, and U.S. Application No. 63/510,993, filed on Jun. 29, 2023, the contents of which are hereby incorporated by reference in their entirety.

### INCORPORATION BY REFERENCE

The Sequence Listing XML file named “Sequence-listings_VP156743-010114”, created on May 9, 2024, and having a size of 25 kilobytes, is incorporated herein by reference in its entirety.

### BACKGROUND OF THE INVENTION

Recombinant AAV (rAAV) vectors are typically produced by replacing the viral coding sequences of the adeno-associated virus (AAV) with a gene of interest (transgene). rAAV vectors are believed to be the most promising viral vector to treat genetic diseases. However, rAAV has a limited cargo capacity of less than 5.0 kb. If the size of the transgene of interest is larger than 5.0 kb, rAAV vectors could not be fully packaged in the AAV capsids, thus cannot be used for efficient gene therapy.

rAAV vector is usually composed of an expression cassette for the therapeutic gene and two ITRs. The expression cassette includes a promoter, a gene of interest (transgene), and a polyA sequence. The shortest polyA sequence available now is around 50 bp. The two ITRs are located at both ends of the expression cassette. Each ITR is 145 bp long, and they are the packaging signals that must be incorporated in the rAAV vector. Thus, the space allocated for a therapeutic gene expression cassette is around 4.71 kb in length.

Some DNA fragments encoding therapeutic proteins are oversized for the rAAV packaging capacity. To keep the expression cassette in the range of efficient packaging, the size of a promoter is important. A short promoter is preferable. In addition, to ensure safety and therapeutic efficiency, a promoter should be liver-specific and drive robust gene expression, since administration of a large amount of rAAV vectors may elicit adverse immune responses. As such, in the context of rAAV gene therapy for hemophilia A (HA), short, liver-specific and robust promoters are urgently needed.

In addition, although some DNA fragments encoding therapeutic proteins are small enough for efficient packaging, the encoded proteins' activity remains low. Consequently, a large amount of rAAV vectors must be injected into the patients to produce sufficient therapeutic proteins. However, administration of a large amount of rAAV vectors may elicit adverse immune responses. Thus, there exists an urgent need to develop robust, tissue-specific promoters and enhancers in order to enhance the activity of the encoded therapeutic proteins.

### SUMMARY OF THE INVENTION

The present invention provides engineered liver-specific enhancers, synthetic promoters containing the enhancers, expression vectors containing the synthetic promoters, as well as methods of using the enhancer or the expression vector thereof to address the need in the field, such as treatment of various genetic diseases or conditions associated with the liver.

In one aspect, the present invention provides an engineered enhancer comprising one or more DNA binding sites for transcription factors, wherein the transcription factor is selected from the group consisting of HNF-4α, HNF-3β, D site-binding protein (DBP), CCAAT enhancer binding protein alpha/beta (C/EBP-α/β), and hepatocyte nuclear factor 1 alpha/beta (HNF-1α/β).

In some embodiments, the DNA binding sites for transcription factor HNF-4α, HNF-3β, DBP, C/EBP-α/β and HNF-1α/β comprise nucleic acid sequences of SEQ ID NOs: 4-8, respectively.

In some embodiments, the engineered enhancer comprises a nucleic acid sequence of SEQ ID NO: 3, SEQ ID NO: 9, SEQ ID NO: 12, or a nucleic acid sequence having at least 70%, 75%, 80%, 85%, 90%, 95%, 99%, or 100% sequence identity to SEQ ID NO: 3, 9, or 12.

In another aspect, the present invention provides a synthetic promoter comprising the engineered enhancer disclosed herein and a nucleic acid sequence of a core promoter.

In some embodiments, the core promoter is a liver-specific promoter.

In some embodiments, the liver-specific promoter is a human α-1 antitrypsin (hAAT) promoter.

In some embodiments, the human α-1 antitrypsin (hAAT) promoter comprises a nucleic acid sequence of SEQ ID NO: 1, SEQ ID NO: 2, or a nucleic acid sequence having at least 80%, 85%, 90%, 95%, 99%, or 100% sequence identity to SEQ ID NO: 1 or 2.

In some embodiments, the synthetic promoter comprises a nucleic acid sequence of SEQ ID NO: 10, SEQ ID NO: 13, or SEQ ID NO: 14, or a nucleic acid sequence having at least 70%, 75%, 80%, 85%, 90%, 95%, 99%, or 100% sequence identity to SEQ ID NO: 10, 13, or 14.

In another aspect, the present invention provides an expression vector comprising the synthetic promoter disclosed herein operably linked to a gene of interest (transgene).

In some embodiments, the expression vector is a plasmid, a recombinant retroviral vector, a recombinant lentiviral vector, a recombinant adenoviral vector, or a recombinant adeno-associated viral vector (rAAV).

In another aspect, the present invention provides a method of treating a genetic disease or condition in a subject in need thereof, comprising administering an expression vector disclosed herein to the subject, thereby expressing a therapeutic protein in the subject's liver.

In some embodiments, the subject is a mammal. Preferably, the mammal is a human.

Examples of the genetic disease or condition associated with the liver include, but not limited to, genetic cholestasis, hemophilia A, hemophilia B, phenylketonuria, hereditary hemochromatosis, tyrosinemia type 1, alpha-1 antitrypsin deficiency, argininosuccinic aciduria, liver cancer, glycogen storage disease, urea cycle disorder, Crigler-Najjar syndrome, familial amyloid polyneuropathy, atypical hemolytic uremic syndrome-1, primary hyperoxaluria type 1, maple syrup urine disease, acute intermittent porphyria, coagulation defects, GSD type1A, homozygous familial hypercholesterolemia, organic acidurias, cystic fibrosis, erythropoietic protoporphyria, Gaucher disease, familial hypercholesterolemia, ornithine and transcarbamylase deficiency.

In another aspect, the present invention provides use of the promoter, or the expression vectors disclosed herein in enhancing the expression level of a transgene in a liver cell, wherein the nucleic acid comprises a transgene operably linked to the promoter.

In another aspect, the present invention provides use of the promoter, or the expression vector, or the pharmaceutical composition provided herein for the manufacture of a medicament for the genetic disease or condition associated with the liver.

In another aspect, the present invention provides a kit comprising the promoter, or the expression vectors, or the pharmaceutical composition disclosed herein.

In some embodiments, the kit further comprises instructions for using the contents of the kit.

The subsequent description and appended claims will provide a better understanding of the features, aspects, and advantages of the present invention.

### BRIEF DESCRIPTION OF THE DRAWINGS

The present invention will now be described by way of non-limitative example with reference to the following figures, in which:

FIG. 1 is a diagram of regulatory elements for driving transgene overexpression in rAAV vector. Enh, Enhancer; Pro, Promoter; Int, Intron; pA, polyA; HCR, complementary sequence of partial coding sequence (CDS) of hepatic control region HCR-1; Ex, proximal element X located in 5′ untranslated region (5′ UTR) of human SERPINA1 (encoding human α-1 antitrypsin) genome; HEx, 66 bp combined enhancer containing HCR and Ex; HLP, 252 bp, a positive control synthetic promoter; hAAT, 219 bp hAAT (human α-1 antitrypsin) promoter; hAATs, 94 bp hAAT promoter.

FIG. 2 is a diagram of engineered enhancers Es, E2, and Em. Transcription factors binding sites (TFBS) \textcircled{1}-\textcircled{5} were obtained from JASPAR. TFBS \textcircled{1}-\textcircled{5} refers to the DNA binding sites of HNF-4α, HNF-3β, DBP, C/EBP-α/β, and HNF-1α/β transcription factors, respectively. Es, 54 bp enhancer was engineered to contain the combination of TFBS \textcircled{1}-\textcircled{2}; Es-2, 52 bp enhancer, was engineered similar to Es but with one thymine and one adenine deletion at the 5′ end and 3′ end of HNF-3β transcription factor binding site, respectively (\textcircled{6}); E2, 111 bp enhancer containing Es and Es-2; Em, another 54 bp enhancer was engineered to contain the DNA binding sites as shown in the diagram, where \textcircled{7} is similar to \textcircled{5}, except the seventh base guanine was converted to cytosine.

FIG. 3 is a diagram of synthetic promoters for driving FVIII-SQ or luciferase overexpression in rAAV vector. HCR, complementary sequence of partial coding sequence (CDS) of hepatic control region HCR-1; Ex, proximal element X located in 5′ untranslated region (5′ UTR) of human SERPINA1 (encoding human α-1 antitrypsin) genome; HLP, 252 bp, a positive control synthetic promoter; HEx, 66 bp combined enhancer containing HCR and Ex; hAATs, 94 bp hAAT promoter; HEx-hAATs, 177 bp synthetic promoter containing 94 bp hAAT promoter and 66 bp HEx enhancer; Es, 54 bp enhancer containing the combination of the DNA binding sites of 5 transcription factors; Es-2, 52 bp enhancer similar to Es but with one thymine and one adenine deletion at the 5′ end and 3′ end of HNF-3β transcription factor binding site, respectively. Es-hAATs, 164 bp synthetic promoter containing 94 bp human α-1 antitrypsin short core promoter and 54 bp enhancer Es; E2-hAATs, 215 bp synthetic promoter containing 94 bp human α-1 antitrypsin short core promoter, 54 bp enhancer Es, and 52 bp enhancer Es-2; Em, another 54 bp enhancer; Em-hAATs, 164 bp synthetic promoter containing 94 bp human α-1 antitrypsin short core promoter and 54 bp enhancer Em.

FIG. 4A-4B show the activities of liver-specific core promoters. Different rAAV vector plasmids were transfected in Huh7 cells on 12-well plates. Supernatants of cells were collected 48 hours post-transfection to measure the activities and protein levels of FVIII-SQ by APTT and ELISA. HLP, 252 bp, a positive control synthetic promoter; HEx, 66 bp combined enhancer containing HCR and Ex; HCR, complementary sequence of partial coding sequence (CDS) of hepatic control region HCR-1; Ex, proximal element X located in 5′ untranslated region (5′ UTR) of human SERPINA1 (encoding human α-1 antitrypsin) genome; hAAT, 219 bp hAAT (human α-1 antitrypsin) promoter; hAATs, 94 bp hAAT promoter; HEx-hAATs, 177 bp synthetic promoter with enhancer HEx. FIG. 4A shows the activities of FVIII-SQ in Huh7 cells transfected with different plasmids. FIG. 4B shows the protein levels of FVIII-SQ in Huh7 cells transfected with different plasmids. *P<0.05, **P<0.01, ***P<0.001 and ns represent not significant between HLP and other promoter groups using a two-tailed Student's t-test.

FIG. 5A-5D show the effect of engineered enhancer on the hAATs promoter. FIG. 5A shows the activities of FVIII-SQ in Huh7 cells. FIG. 5B shows the protein levels of FVIII-SQ in Huh7 cells. Transcription factors binding sites (TFBS) were obtained from JASPAR. Different rAAV vector plasmids were transfected in Huh7 cells on 12-well plates. Supernatants of cells were collected 48 hours post-transfection to measure the activities and protein levels of FVIII-SQ by APTT and ELISA. HLP, 252 bp, a positive control synthetic promoter; E2, 111 bp enhancer containing Es and Es-2; Es, 54 bp enhancer combined with DNA binding sites of transcription factors; Es-2, 52 bp enhancer similar to Es but with one thymine and one adenine deletion at the 5′ end and 3′ end of HNF-3β transcription factor binding site, respectively; hAATs, 94 bp human α-1 antitrypsin promoter; Es-hAATs, 164 bp synthetic promoter with enhancer Es; E2-hAATs, 215 bp synthetic promoter containing 94 bp human α-1 antitrypsin short promoter and enhancer Es and enhancer Es-2. FIG. 5C shows the activities of FVIII-SQ in factor VIII deficient mice. FIG. 5D shows the protein levels of FVIII-SQ in factor VIII deficient mice. Around 8-10 weeks old factor VIII deficient mice were injected different rAAV vector plasmids via hydrodynamic injection. Plasma of mice were collected 48 hours post-injection to measure the activities and protein levels of FVIII-SQ by APTT and ELISA. *P<0.05, **P<0.01, ***P<0.001 and ns represent not significant between HLP and other promoter groups (or between other two promoter groups) using a two-tailed Student's t-test.

FIG. 6 shows the effects of different enhancers on the hAATs promoter. Different rAAV vector plasmids were transfected in Huh7 cells on 12-well plates. Cells were collected 24 hours post-transfection to measure the activities of luciferase. HLP, 252 bp, a positive control synthetic promoter; HEx-hAATs, 177 bp synthetic promoter with 66 bp enhancer HEx; Es-hAATs, 164 bp synthetic promoter containing 94 bp human α-1 antitrypsin short promoter and 54 bp enhancer Es; E2-hAATs, 215 bp synthetic promoter containing 94 bp human α-1 antitrypsin short promoter and 54 bp enhancer Es and 52 bp Es-2; Em-hAATs, 164 bp synthetic promoter containing 94 bp human α-1 antitrypsin short promoter and 54 bp enhancer Em. *P<0.05, **P<0.01, ***P<0.001, ****P<0.0001 and ns represent not significant between HLP and other promoter groups (or between other two promoter groups) using a two-tailed Student's t-test.

### DETAILED DESCRIPTION OF THE INVENTION

References to specific features of the invention are made in the Summary Section above, and the Detailed Description Section and claims below. It is to be understood that the disclosure of the invention in this specification includes all possible combinations of such specific features. For example, where a specific feature is disclosed in the context of a particular aspect or embodiment of the invention or in a particular claim, the feature can also be used, to the extent possible, in combination with and/or in the context of other particular aspects and embodiments of the invention, and in the invention generally.

Gene therapy is a promising method to treat genetic diseases. In gene therapy, a gene of interest is introduced into one or more recipient cells, and expression of the introduced gene in the recipient cell affects the cell's function and results in a therapeutic effect in a subject. rAAV vectors are believed to be the most promising viral vector to be used in gene therapy. However, rAAV has a limited cargo capacity of less than 5.0 kb.

Except for other necessary elements, the space for the length of a promoter and a therapeutic gene is around 4.71 kb. Thus, the shorter the promoter, the more space for the therapeutic gene. Also, a liver-specific promoter is preferable, as it prevents gene expression in organs other than liver, thereby, ensuring the safety of the therapy. In addition, a promoter would be more efficient if it drives robust gene expression, thereby, ensuring the therapeutic efficiency.

In the first aspect, the present invention provides an engineered enhancer comprising one or more DNA binding sites for transcription factors.

As used herein, an “enhancer” is a nucleic acid sequence that increases the rate of transcription by increasing the activity of a promoter. An “engineered enhancer” refers to a man-made enhancer that is not occurring naturally. An “engineered promoter” is sometimes called an “artificial promoter” or “synthetic promoter.”

Transcription factors are proteins that bind to specific DNA sequences and thereby control the transfer (or transcription) of genetic information from DNA to RNA. The “DNA binding sites” used herein refer to the specific DNA sequences that transcription factors bind to.

In some embodiments, the transcription factor is selected from the group consisting of hepatocyte nuclear factor-4α (HNF-4α), hepatocyte nuclear factor 3-beta (HNF-3β), D site-binding protein (DBP), CCAAT enhancer binding protein alpha (C/EBP-α), CCAAT enhancer binding protein beta (C/EBP-B), hepatocyte nuclear factor 1 alpha (HNF-1α), and hepatocyte nuclear factor 1 beta (HNF-1B).

HNF-4a is encoded by the HNF4A gene and is a nuclear transcription factor that binds DNA as a homodimer. It controls the expression of several genes, including hepatocyte nuclear factor 1 alpha, a transcription factor which regulates the expression of several hepatic genes.

HNF-3β is encoded by the FOXA2 gene and is a member of the forkhead class of DNA-binding proteins. These hepatocyte nuclear factors are transcriptional activators for liver-specific genes such as albumin and transthyretin.

DBP is a transcriptional activator that recognizes and binds to the sequence 5′-RTTAYGTAAY-3′ found in the promoter of genes such as albumin, CYP2A4 and CYP2A5.

C/EBP-α contains a basic leucine zipper (bZIP) domain and recognizes the CCAAT motif in the promoters of target genes. It functions in homodimers and heterodimers with CCAAT/enhancer-binding proteins β and γ. Activity of this protein can modulate the expression of genes involved in cell cycle regulation as well as in body weight homeostasis.

C/EBP-β is a bZIP transcription factor that can bind as a homodimer to certain DNA regulatory regions. It can also form heterodimers with the related proteins C/EBP-α, C/EBP-δ, and C/EBP-γ.

HNF-1α is encoded by the HNF1A gene and is a transcription factor that is highly expressed in the liver and is involved in the regulation of the expression of several liver-specific genes.

HNF-1β is encoded by the HNF1B gene and is a protein of the homeobox-containing basic helix-turn-helix family. The HNF1B protein is believed to form heterodimers with another member of this transcription factor family, HNF-1α.

In some embodiments, the DNA binding sites of HNF-4α, HNF-3β, DBP, C/EBP-α/β and HNF-1α/β comprise sequences of SEQ ID NOs: 4-8, respectively.
SEQ ID NO: 4: HNF-4 a transcription factor DNA binding site (TFBS)
TGGACTTTGCACT
SEQ ID NO: 5: HNF-3 B TFBS
TGTAAACA
SEQ ID NO: 6: DBP TFBS
ATTACGTAAC
SEQ ID NO: 7: C/EBP-α/β TFBS
ATTGCACAAT
SEQ ID NO: 8: HNF-1α/β TFBS
GTTAATGATTAAC

In some embodiments, the engineered enhancer comprises a sequence of SEQ ID NO:

3, SEQ ID NO: 9, SEQ ID NO: 12, or a nucleic acid sequence having at least 70%, 75%, 80%, 85%, 90%, 95%, 99%, or 100% sequence identity to SEQ ID NO: 3, SEQ ID NO: 9, or SEQ ID NO: 12.

Es enhancer (54 bp)
SEQ ID NO: 3:
TGGACTTTGCACTTGTAAACAATTACGTAACATTGCACAATGTTAATGAT
TAAC

The Es enhancer is 54 bp and contains, from 5′ end to 3′ end, the DNA binding sites of HNF-4α, HNF-3β, DBP, C/EBP-α/β and HNF-1α/β.

Em enhancer
SEQ ID NO: 9:
TGGACTTTGCACTATTGCACAATTGTAAACAGTTAATCATTAACATTACG
TAAC

The Em enhancer is 54 bp and contains, from 5′ end to 3′ end, the DNA binding sites of HNF-4α, C/EBP-α/β, HNF-3β, HNF-1α/β (the seventh base guanine was converted to cytosine), and DBP.

Es-2 enhancer
SEQ ID NO: 12:
TGGACTTTGCACTGTAAACATTACGTAACATTGCACAATGTTAATGATTA
AC

The Es-2 enhancer is 52 bp and similar to the Es enhancer, except that two nucleotides have been deleted from the Es-2 enhancer. Specifically, one thymine and one adenine have been deleted at the 5′ end and 3′ end of HNF-3β transcription factor binding site, respectively.

In another aspect, the present invention provides a synthetic promoter comprising the engineered enhancer disclosed herein and a nucleic acid sequence of a core promoter.

As used herein, a “synthetic promoter” is a stretch of DNA comprising a core promoter and a combination of heterologous upstream regulatory elements (cis-motifs or transcription factor-binding sites). A “synthetic promoter” is sometimes called an “engineered promoter” or “artificial promoter.” The core promoter (also known as the minimal-region) usually contains a TATA-box necessary for recruiting RNA polymerase II and the assembly of general transcription factors to form the preinitiation complex. A synthetic promoter may comprise, for example, regions of known promoters, regulatory elements, transcription factor binding sites, enhancer elements, repressor elements, and the like.

In some embodiments, the core promoter is a liver-specific promoter.

A liver-specific promoter primarily directs transgene expression in the liver, but it may also lead to transgene expression in other tissues or organs at lower levels. Use of a liver-specific promoter in the expression cassette can restrict unwanted transgene expression in other tissues and facilitate persistent transgene expression in the liver.

In some embodiments, the liver-specific promoter is a human α-1 antitrypsin (hAAT) promoter.

In human, SERPINA1 gene encodes hAAT, which is produced in the liver and then transported throughout the body via the blood. Thus, the hAAT promoter is believed to drive gene expression specifically in the liver.

In some embodiments, the human α-1 antitrypsin (hAAT) promoter comprises a nucleic acid sequence of SEQ ID NO: 1, SEQ ID NO: 2, or a nucleic acid sequence having at least 80%, 85%, 90%, 95%, 99%, or 100% sequence identity to SEQ ID NO: 1 or 2.

hAAT promoter (219 bp)
SEQ ID NO: 1:
TGACTCCTTTCGGTAAGTGCAGTGGAAGCTGTACACTGCCCAGGCAAAGC
GTCCGGGCAGCGTAGGCGGGCGACTCAGATCCCAGCCAGTGGACTTAGCC
CCTGTTTGCTCCTCCGATAACTGGGGTGACCTTGGTTAATATTCACCAGC
AGCCTCCCCCGTTGCCCCTCTGGATCCACTGCTTAAATACGGACGAGGAC
AGGGCCCTGTCTCCTCAGC
hAATs promoter (94 bp)
SEQ ID NO: 2:
GTGACCTTGGTTAATATTCACCAGCAGCCTCCCCCGTTGCCCCTCTGGAT
CCACTGCTTAAATACGGACGAGGACAGGGCCCTGTCTCCTCAGC

Each of SEQ ID NO: 1 and SEQ ID NO: 2 contains an upstream sequence of the human SERPINA1 gene. The promoters having the sequences of SEQ ID NO: 1 and SEQ ID NO: 2 are named as hAAT and hAATs, respectively. These promoters are selected for construction of synthetic promoters of some embodiments.

In some embodiments, the synthetic promoter comprises a nucleic acid sequence of SEQ ID NO: 10, SEQ ID NO: 13, or SEQ ID NO: 14, or a nucleic acid sequence having at least 70%, 75%, 80%, 85%, 90%, 95%, 99%, or 100% sequence identity to SEQ ID NO: 10, 13, or 14.

Es-hAATs synthetic promoter
SEQ ID NO: 10:
TGGACTTTGCACTTGTAAACAATTACGTAACATTGCACAATGTTAATGAT
TAACTTAAGCGTCGACACGCGTGACCTTGGTTAATATTCACCAGCAGCCT
CCCCCGTTGCCCCTCTGGATCCACTGCTTAAATACGGACGAGGACAGGGC
CCTGTCTCCTCAGC

The Es-hAATs synthetic promoter is 164 bp long and includes the 54 bp enhancer Es and the 94 bp promoter hAATs, connected by necessary enzyme digestion sites.

E2-hAATs synthetic promoter
SEQ ID NO: 13:
TGGACTTTGCACTTGTAAACAATTACGTAACATTGCACAATGTTAATGAT
TAACTTAAGTGGACTTTGCACTGTAAACATTACGTAACATTGCACAATGT
TAATGATTAACGTCGACACGCGTGACCTTGGTTAATATTCACCAGCAGCC
TCCCCCGTTGCCCCTCTGGATCCACTGCTTAAATACGGACGAGGACAGGG
CCCTGTCTCCTCAGC

The E2-hAATs synthetic promoter is 215 bp long and includes the 54 bp engineered enhancer Es, the 52 bp enhancer Es-2, and the 94 bp promoter hAATs, connected by necessary enzyme digestion sites.

Em-hAATs synthetic promoter
SEQ ID NO: 14:
TGGACTTTGCACTATTGCACAATTGTAAACAGTTAATCATTAAcATTACG
TAACTTAAGCGTCGACACGCGTGACCTTGGTTAATATTCACCAGCAGCCT
CCCCCGTTGCCCCTCTGGATCCACTGCTTAAATACGGACGAGGACAGGGC
CCTGTCTCCTCAGC

The Em-hAATs synthetic promoter is 164 bp long and includes the 54 bp enhancer Em and the 94 bp promoter hAATs, connected by necessary enzyme digestion sites.

In another aspect, the present invention provides an expression vector comprising the synthetic promoter disclosed herein operably linked to a gene of interest.

The term “operably linked” means that the regulatory sequences necessary for expression of a coding sequence are placed in the DNA molecule in the appropriate positions relative to the coding sequence so as to effect expression of the coding sequence.

In some embodiments, the expression vector is a plasmid, a recombinant retroviral vector, a recombinant lentiviral vector, a recombinant adenoviral vector, or a recombinant adeno-associated viral vector (rAAV). In some preferable embodiments, the expression vector is an rAAV.

Human adeno-associated virus (AAV) is a non-pathogenic parvovirus that only productively replicates in cells co-infected by a helper virus, usually adenovirus or herpes virus. The virus has a wide host range and can productively infect many cell types from a variety of animal species. Nevertheless, AAV has not been implicated in any human or animal disease.

AAV binds to cells via a heparan sulfate proteoglycan receptor. Once attached, AAV entry is dependent upon the presence of a co-receptor, either the fibroblast growth factor receptor or αvβ5 integrin molecule. In infected cells, the incoming AAV single-stranded DNA (ssDNA) is converted to double-stranded transcriptional template. Cells infected with AAV and a helper virus will undergo productive replication of AAV prior to cell lysis, which is induced by the helper virus rather than AAV. Helper virus encodes proteins or RNA transcripts which are transcriptional regulators and are involved in DNA replication or modify the cellular environment in order to permit efficient viral production.

Recombinant AAV (rAAV) vectors are typically produced by replacing the viral coding sequences with transgenes of interest. These vectors have been shown to be highly efficient for gene transfer and expression at a number of different sites in vitro and in vivo. They have consistently mediated stable expression and have been safe in studies performed in the respiratory tract, the central nervous system, skeletal muscle, liver, and eye. The efficiency of rAAV-mediated transduction has increased as the titer and purity of rAAV preparations has improved.

The inverted terminal repeats (ITRs) from the AAV genome are the only viral sequences required in cis to generate rAAV vectors. Recombinant constructs containing two ITRs bracketing a gene expression cassette of ˜5 kb are converted into a ssDNA vector genome and packaged into AAV particles in the presence of AAV rep and cap gene products and helper functions. Methods or production and purification of rAAV are known in the art.

One of the genes of interest can be the FVIII gene, which is located on the X chromosome and encodes Factor VIII (FVIII). FVIII is one of the main factors for coagulation cascade. The loss-of-function mutation of FVIII causes a genetic disease called Hemophilia A (HA). The occurrence rate for HA is 1/5000 in males.

The common treatment for HA is replacement therapy. Concentrates of factor VIII are slowly dripped or injected into a vein of HA patients. These infusions serve to replace the deficient or low levels of factor VIII in patients with HA. However, this replacement therapy may generate inhibitors of the injected or acquired factor VIII, leading to the failure of this replacement therapy.

An alternative therapy for hemophilia A is gene therapy based on rAAV vectors. The rAAV vectors allow long-term, stable expression of transgenes in vivo for therapeutic purposes. The coding region of FVIII is 7035 bp long and can be divided into 6 domains, namely, A1, A2, B, A3, C1, C2. For rAAV vectors to be efficiently packaged into adeno-associated virus (AAV) capsids, the size of the expression cassette containing a therapeutic gene should generally not exceed 5 kb.

Due to the limitation of AAV packaging capacity, the full-length FVIII coding region will not be effectively packaged into AAV vectors. To overcome this problem, researchers must reduce the size of the coding region of FVIII. Previous studies have shown that B domain of FVIII (908 aa) can be replaced with SQ domain (14 aa), which does not seem to be required for FVIII clotting activity FVIII. This engineered FVIII is known as FVIII-SQ and has 6 domains, i.e., A1, A2, SQ, A3, C1, and C2 domains. A1, A2, and SQ domains form the heavy chain and A3, C1, C2 form the light chain of FVIII-SQ. The nucleotide encoding the FVIII-SQ is 4374 bp, which allows it to be efficiently inserted into rAAV vectors.

However, even when the expression cassette is around 5 kb, many packaged rAAV vectors remain incomplete and defective, and these defective rAAV vectors cannot produce functional FVIII. To address this limitation, a large amount of rAAV vectors need to be injected to HA patients to produce sufficient functional FVIII. However, administration of a large amount of rAAV vectors may elicit adverse immune responses.

To address these limitations, it is essential to maintain the size of both the promoter and the polyA tail as minimal as possible. In some embodiments, the synthetic promoters disclosed herein are used to direct the expression of FVIII-SQ or other engineered FVIII fragments. Due to the reduced size of these synthetic promoters, the expression cassette exhibits higher packaging rate.

In another aspect, the present invention provides pharmaceutical compositions for delivering a transgene described herein to a subject, including a human subject. In some embodiments, the composition comprises any of the nucleic acids or vectors described herein. In some embodiments, pharmaceutical compositions disclosed herein comprise any vector disclosed herein and one or more pharmaceutically acceptable carrier. In some embodiments, the composition comprises any of the AAV vectors described herein. In some embodiments, pharmaceutical compositions disclosed herein comprise any AAV vectors disclosed herein and one or more pharmaceutically acceptable carrier.

Although the descriptions of pharmaceutical compositions provided herein, e.g., AAV vectors, are principally directed to pharmaceutical compositions that are suitable for administration to humans, a skilled artisan in the field would understand that such compositions are generally suitable for administration to any other animals, e.g., to non-human animals, or non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are to be administered to humans.

In another aspect, the present invention provides a method of treating a genetic disease or condition in a subject in need thereof, comprising administering an expression vector disclosed herein to the subject, thereby expressing a therapeutic protein in the subject's liver.

In some embodiments, the subject is a mammal. In some embodiments, the subject is a human.

Examples of the genetic disease or condition associated with the liver include, but are not limited to, genetic cholestasis, hemophilia A, hemophilia B, phenylketonuria, hereditary hemochromatosis, tyrosinemia type 1, alpha-1 antitrypsin deficiency, argininosuccinic aciduria, liver cancer, glycogen storage disease, urea cycle disorder, Crigler-Najjar syndrome, familial amyloid polyneuropathy, atypical hemolytic uremic syndrome-1, primary hyperoxaluria type 1, maple syrup urine disease, acute intermittent porphyria, coagulation defects, GSD type1A, homozygous familial hypercholesterolemia, organic acidurias, cystic fibrosis, erythropoietic protoporphyria, Gaucher disease, familial hypercholesterolemia, ornithine and transcarbamylase deficiency.

In some embodiments, methods provided herein can be used to treat a genetic disease or condition in a subject in need, comprising administering an expression vector disclosed herein to the subject, thereby expressing a therapeutic protein in the subject's liver.

In another aspect, the present invention provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the pharmaceutical compositions or vectors of the present disclosure may be included in a kit. In some embodiments, kits can further include reagents and/or instructions for creating and/or synthesizing compounds and/or pharmaceutical compositions of the present disclosure. In some embodiments, kits can also include one or more buffers. In some embodiments, kits of the disclosure can include components for making protein or nucleic acid arrays or libraries and thus, may include, for example, solid supports.

In some embodiments, kit components can be packaged either in aqueous media or in lyophilized form. The container means of the kits will generally include at least one vial, test tube, flask, bottle, syringe or other container means, into which a component can be placed, and suitably aliquoted. Where there is more than one kit component, (labeling reagent and label may be packaged together), kits can also generally contain second, third or other additional containers into which additional components may be separately placed. In some embodiments, kits can also include a second container means for containing sterile, pharmaceutically acceptable buffers and/or other diluents. In some embodiments, various combinations of components can be included in one or more vials. Kits of the present disclosure can also typically include means for containing compounds and/or pharmaceutical compositions of the present disclosure, e.g., proteins, nucleic acids, and any other reagent containers in close confinement for commercial sale. Such containers can include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components can be provided as dried powder(s). When reagents and/or components are provided as dry powders, such powders can be reconstituted by the addition of suitable volumes of solvent. In some embodiments, it is envisioned that solvents can also be provided in another container means. In some embodiments, kits can include instructions for employing kit components as well the use of any other reagent not included in the kit. Instructions can include variations that may be implemented.

### Definition

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, the terms “individual,” “patient,” or “subject” are used Interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “S′ end” when its 5′ phosphate is not linked to the 3′ oxygen of a mononucleotide pentose ring. The other end of a polynucleotide is referred to as the “3′ end” when its 3′ oxygen is not linked to a S′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

The term “promoter region” or “promoter” refers to a region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell. For example, a liver-specific promoter is a promoter that directs/initiates transcription in liver tissue to a substantially greater extent than other tissue types.

The term “enhancer” refers to a nucleic acid sequence that increases the rate of transcription by increasing the activity of a promoter.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

The term “vector” refers to a small carrier DNA molecule into which a DNA sequence can be inserted for introduction into a host cell where it will be replicated. An “expression vector” is a specialized vector that contains a gene or nucleic acid sequence with the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g., promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, the term “percent identical” is used herein with reference to comparisons among nucleic acid or amino acid sequences. It is defined as the percentage of nucleotides or amino acid residues in a candidate sequence that are identical with the nucleotides or amino acid residues in a specific sequence, after aligning the sequences and Introducing gaps, if necessary, to achieve the maximum percent sequence identity. Nucleic acid and amino acid sequences are often compared using computer programs that align sequences of nucleic or amino acids thus defining the differences between the two. Comparisons of nucleic acid or amino acid sequences may be performed in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The phrase “sequence identity” refers to the identity (I.e., being identical) or similarity between two or more nucleic acid sequences, or two or more amino acid sequences, which is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G] dUTP, [TAMRA] dUTP, [R110] dCTP, [R6G] dCTP, [TAMRA] dCTP, [JOE] ddATP, [R6G] ddATP, [FAM] ddCTP, [R110] ddCTP, [TAMRA] ddGTP, [ROX] ddTTP, [dR6G] ddATP, [dR110] ddCTP, [dTAMRA] ddGTP, and [dROX] ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

The term “cDNA (complementary DNA)” refers to a piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

The term “gene,” as used herein, refers to a nucleic acid (e.g., DNA such as genomic DNA and cDNA) and its corresponding nucleotide sequence that is involved in encoding an RNA transcript. The term used herein with reference to genomic DNA includes intervening, non-coding regions as well as regulatory regions and can include 5′ and 3′ ends. In some uses, the term encompasses the transcribed sequences, including 5′ and 3′ untranslated regions (5′ -UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

Transcription factor (TF) is a protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element.

Gene therapy involves the introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects the function of the recipient cell.

Inverted terminal repeat (ITR) refers to symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors. The term “Control” as used herein refers to a reference standard.

Hemophilia is a blood coagulation disorder caused by a deficient clotting factor activity, which decreases hemostasis. Severe forms result when the concentration of clotting factor is less than about 1% of the normal concentration of the clotting factor in a normal subject. In some subjects, hemophilia is due to a genetic mutation which results in impaired expression of a clotting factor. In others, hemophilia is an auto-immune disorder, referred to as acquired hemophilia, in which the antibodies which are generated against a clotting factor in a subject result in decreased hemostasis.

Hemophilia A results from a deficiency of functional clotting Factor VIII, while hemophilia B results from a deficiency of functional clotting Factor IX. These conditions which are due to a genetic mutation are caused by an inherited sex-linked recessive trait with the defective gene located on the X chromosome, and this disease is therefore generally found only in males. The severity of symptoms can vary with this disease, and the severe forms become apparent early on. Bleeding is the hallmark of the disease and typically occurs when a male infant is circumcised. Additional bleeding manifestations make their appearance when the infant becomes mobile. Mild cases may go unnoticed until later in life when they occur in response to surgery or trauma. Internal bleeding may happen anywhere, and bleeding into joints is common.

“Factor VIII deficiency,” as used herein, includes deficiency in clotting activity caused by production of defective factor VIII, by inadequate or no production of factor VIII, or by partial or total inhibition of factor VIII by inhibitors. Hemophilia A is a type of factor VIII deficiency resulting from a defect in an X-linked gene and the absence or deficiency of the factor VIII protein it encodes.

The terms “derivative,” “variant” and “fragment” when used herein with reference to a polypeptide, refers to a polypeptide related to a wild type of polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a referenced polypeptide.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “pharmaceutical composition” refers to a mixture of the expression vectors disclosed herein or the rAAV vectors disclosed herein with other chemical components, such as diluents or carriers. The pharmaceutical composition facilitates administration of the compound to an organism. Pharmaceutical compositions will generally be tailored to the specific intended route of administration. A pharmaceutical composition is suitable for human and/or veterinary applications.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising rAAV vectors, that is sufficient to result in a desired activity upon administration to a subject in need thereof. The term “therapeutically effective” can refer to a quantity of a composition that is sufficient to delay the manifestation, arrest the progression, relieve or alleviate at least one symptom of a disorder treated by the methods of the present disclosure.

A “therapeutic effect” may occur if there is a change in the condition being treated. The change may be positive or negative. For example, a “positive effect” may correspond to an increase in the number of activated T-cells in a subject. In another example, a ‘negative effect’ may correspond to a decrease in the amount or size of a tumor in a subject. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. Similarly, a method of the present disclosure may comprise administering to a subject a number of cells that are “therapeutically effective.” The term “therapeutically effective” should be understood to have a definition corresponding to ‘having a therapeutic effect.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Designing and Obtaining Liver-Specific Enhancers and Testing their Effects on Promoters

This example describes how the liver-specific enhancers were designed and obtained, and their effects on promoters were tested. Specifically, synthetic promoters, each comprised of a short core promoter and an engineered enhancer, were utilized to drive expression of a B-domain deleted FVIII (FVIII-SQ) in Huh7 cells and mice, or luciferase overexpression in Huh7 cells. The activities and protein levels of FVIII-SQ and luciferase activities were measured to compare the activities of synthetic promoters that have different combination of core promoters and enhancers. Results show that the synthetic promoters were shorter but had significant higher promoting activities than the 252 bp HLP liver-specific promoter. Methods

### Specific Enhancers Design

Several transcription factors including hepatocyte nuclear factor 1 alpha/beta (HNF-1α/β), HNF-3β, HNF-4α, CCAAT enhancer binding protein alpha/beta (C/EBP-α/β), and D site-binding protein (DBP) were selected and used in this invention.

In this invention, DNA binding sites of the above-mentioned transcription factors (TFBS) were arranged in combination to form a 54 bp engineered enhancer (Es, SEQ ID NO: 3), which was then added upstream of a core promoter to enhance transgene overexpression. The sequences of TFBS of HNF-4α, HNF-3β, DBP, C/EBP-α/β and HNF-1α/β selected in this invention are shown in SEQ ID NOs: 4-8, respectively.

Another 54 bp engineered enhancer (Em, SEQ ID NO: 9) was designed to remove ATG triple nucleotides by rearranging the TFBS of the above-mentioned transcription factors and replacing the seventh base guanine in the sequences of TFBS of HNF-1α/β with cytosine.

A third enhancer, Es-2 (SEQ ID NO: 12), is designed, which is 52 bp and similar to the Es enhancer, except two nucleotides have been deleted from the Es-2 enhancer. Specifically, one thymine and one adenine have been deleted at the 5′ end and 3′ end of HNF-3β transcription factor binding site, respectively.

### Obtaining Liver-Specific Core Promoters

Conservation of sequences between Homo sapiens and Mus musculus, and the position of TATA box related to the transcription start site (TSS) were taken into consideration of choosing promoters in this invention.

Two core promoters, upstream sequences of human SERPINA1 gene (encoding human α-1 antitrypsin, hAAT), called the 219 bp hAAT promoter and the 94 bp hAATs promoter, were selected for in vitro construction of synthetic promoters. The 219 bp hAAT promoter (SEQ ID NO: 1) contains the complementary genome sequence of Chr14: 94388594-94388812 (NC_000014.9). The 94 bp hAATs promoter (SEQ ID NO: 2) contains the complementary genome sequence of Chr14: 94388594-94388687.

Construction of rAAV Vector Plasmids

A liver specific HLP promoter was chosen as a positive control promoter according to the reference (Blood. 2013 Apr. 25; 121 (17): 3335-3344.) and the patent EP 2,698,163 B1. HLP promoter was synthesized and cloned into pUC-HLP plasmid by GENERAL BIOSYSTEMS (Anhui, China).

Promoters hAAT and hAATs were obtained by polymerase chain reaction (PCR) using the genomic DNA from Huh7 cells as the template. Promoter HLP was obtained by PCR using pUC-HLP plasmid as the template. After Mlu I and Nhe I double restriction enzymes digestion, these promoters were cloned into the pssAAV-TTR-FVIII-SQ, the rAAV vector backbone, to construct recombinant vectors including pssAAV-hAAT-FVIII-SQ, pssAAV-hAATs-FVIII-SQ and pssAAV-HLP-FVIII-SQ. pssAAV-TTR-FVIII-SQ is a plasmid expression vector contains a TTR promoter, an intron, FVIII-SQ coding sequences and a polyA tail.

A single chain oligonucleotide with the 54 bp engineered enhancer(Es) and several enzymatic cleavage sites under 90 nucleotides was synthesized by GENEWIZ (Suzhou, China). Its complementary oligonucleotide was also synthesized by GENEWIZ. These two oligonucleotides were combined to form a DNA fragment, which has sticky ends of the Mlu I enzyme digestion site at both ends. Thereafter, this DNA fragment was cloned into pssAAV-hAATs-FVIII-SQ vector to obtain the pssAAV-Es-hAATs-FVIII-SQ vector. The sequence of the Es-hAATs synthetic promoter is shown in SEQ ID NO: 10, which contains the 54 bp engineered enhancer(Es) and the 94 bp hAATs promoter.

Similarly, a fragment containing a 34 bp HCR (hepatic control region) and a 32 bp proximal enhancer element from HLP promoter was cloned into pssAAV-Es-hAATs-FVIII-SQ vector following HindIII and AfIII double enzymes digestion to construct the pssAAV-HEx-hAATs-FVIII-SQ vector. The sequence of the HEx-hAATs synthetic promoter is shown below in SEQ ID NO: 11, which contains the 34 bp HCR (hepatic control region), the 32 bp proximal enhancer element from HLP promoter, and the 94 bp hAATs promoter.

HEx-hAATs synthetic promoter
SEQ ID NO: 11
TGTTTGCTGCTTGCAATGTTTGCCCATTTTAGGGTGGACACAGGACGCTG
TGGTTTCTGAGCCAGGcttaagcgtcgacacgcGTGACCTTGGTTAATAT
TCACCAGCAGCCTCCCCCGTTGCCCCTCTGGATCCACTGCTTAAATACGG
ACGAGGACAGGGCCCTGTCTCCTCAGC

DNA fragment of engineered enhancer Es-2 (SEQ ID NO: 12, similar to Es but with one thymine and one adenine deletion at the 5′ end and 3′ end of HNF-3β transcription factor binding site, respectively) was formed from two complementary oligonucleotides that were synthesized by GENEWIZ. Subsequently, Es-2 was cloned into pssAAV-Es-hAATs-FVIII-SQ vector backbone following the AflII and Sal I double enzymes digestion to construct pssAAV-E2-hAATs-FVIII-SQ vector. The sequence of the E2-hAATs synthetic promoter is shown in SEQ ID NO: 13, which contains the engineered enhancer Es, the engineered enhancer Es-2, and the 94 bp hAATs promoter.

The vector pssAAV-HLP-FVIII-SQ was digested by Mlu I and Nhe I double enzymes, then HLP fragment was cloned into the pssAAV-MSP-luciferase vector backbone followed by HindIII and Spe I digestions to obtain pssAAV-HLP-luciferase. pssAAV-MSP-luciferase is a plasmid expression vector contains an MSP promoter, an intron, luciferase coding sequences and a BGH polyA tail. Similarly, fragments of HEx-hAATs, Es-hAATs and E2-hAATs obtain from pssAAV-HEx-hAATs-FVIII-SQ, pssAAV-Es-hAATs-FVIII-SQ and pssAAV-E2-hAATs-FVIII-SQ by HindIII and Nhe I double enzymes digestion were cloned into pssAAV-MSP-luciferase vector backbone followed by HindIII and Spe I digestions, to obtain pssAAV-HEx-hAATs-luciferase, pssAAV-Es-hAATs-luciferase, and pssAAV-E2-hAATs-luciferase vectors, respectively.

DNA fragment of the engineered enhancer Em (SEQ ID NO: 9) was formed from two complementary oligonucleotide synthesized by GENEWIZ. Then Em was cloned into pssAAV-Es-hAATs-luciferase vector backbone digested by HindIII and AfIII double enzymes to obtain pssAAV-Em-hAATs-luciferase vector. The sequence of the Em-hAATs synthetic promoter is shown in SEQ ID NO: 14, which contains the engineered enhancer Em and the 94 bp hAATs promoter.

All primers used for vector plasmids construction were listed in Table 1 (SEQ ID NOS: 15-27).

TABLE 1
Primers for construction of the core and synthetic promoters

| SEQ ID NO: | Primer name | Primer sequence (5-3′) |
|---|---|---|
| 15 | HLP-Mlul-F | ACCACGCGTGTTTGCTGCTTGCAATGTTTGCC |
| 16 | HLP-Nhel-R | ACCGCTAGCGATTCACTGTCCCAGGTCAG |
| 17 | hAAT-Mlul-F | ACCACGCGTGACTCCTTTCGGTAAGTGCAG |
| 18 | hAAT-Nhel-R | ACCGCTAGCTGAGGAGACAGGGCCCTGTCC |
| 19 | hAATs-Mlul-F | ACCACGCGTGACCTTGGTTAATATTCACC |
| 20 | HCR-Ex-HindIII-F | AGCTTGTTTGCTGCTTGCAATGTTTGCCCATTTTAGGGT |
| | | GGACACAGGACGCTGTGGTTTCTGAGCCAGGC |
| 21 | HCR-Ex-AflII-R | TTAAGCCTGGCTCAGAAACCACAGCGTCCTGTGTCCACC |
| | | CTAAAATGGGCAAACATTGCAAGCAGCAAACA |
| 22 | Es-Mlul-F | CGCGTAAGCTTGGACTTTGCACTTGTAAACAATTACGTA |
| | | ACATTGCACAATGTTAATGATTAACTTAAGCGTCGACA |
| 23 | Es-Mlul-R | CGCGTGTCGACGCTTAAGTTAATCATTAACATTGTGCAA |
| | | TGTTACGTAATTGTTTACAAGTGCAAAGTCCAAGCTTA |
| 24 | Es-2-AflII-F | TTAAGTGGACTTTGCACTGTAAACATTACGTAACATTGC |
| | | ACAATGTTAATGATTAACG |
| 25 | Es-2-Sall-R | TCGACGTTAATCATTAACATTGTGCAATGTTACGTAATG |
| | | TTTACAGTGCAAAGTCCAC |
| 26 | Em-HindIII-F | AGCTTGGACTTTGCACTATTGCACAATTGTAAACAGTTA |
| | | ATCATTAACATTACGTAAC |
| 27 | Em-AflII-R | TTAAGTTACGTAATGTTAATGATTAACTGTTTACAATTG |
| | | TGCAATAGTGCAAAGTCCA |

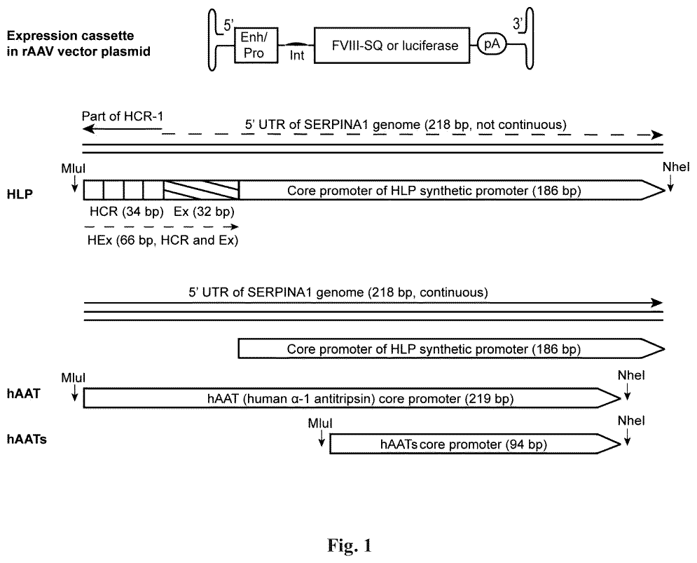

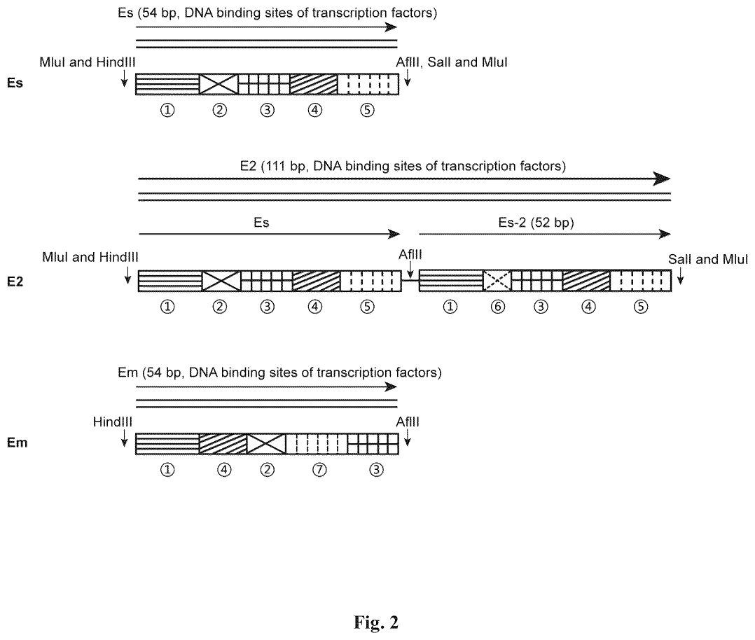

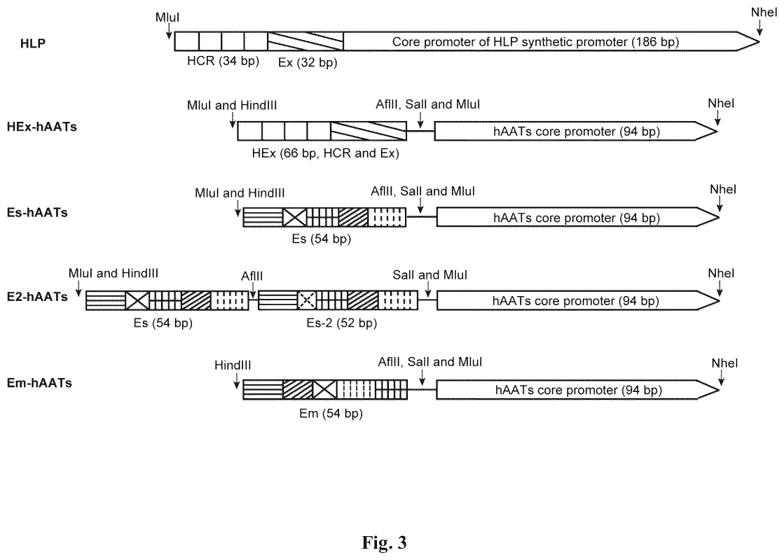

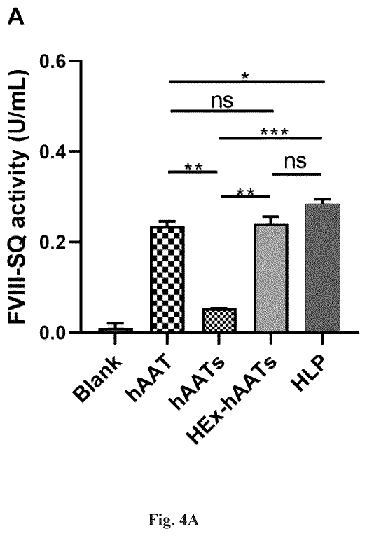

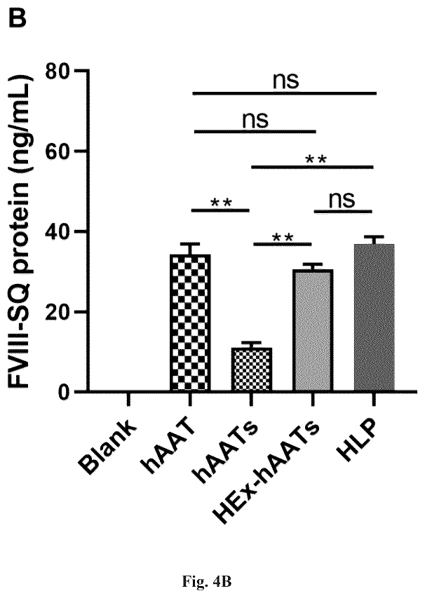

FIG. 1 shows the diagrams of the positive control liver-specific promoter HLP (Blood. 2013 Apr. 25; 121 (17): 3335-3344.) and the core promoters hAAT and hAATs for driving FVIII-SQ overexpression in rAAV vector plasmid. FIG. 2 shows the diagrams of engineered enhancers Es, E2, and Em. FIG. 3 shows the diagrams of HLP promoter and several synthetic promoters.

### Cell Culture and Transfection

Huh7 cells were obtained from ATCC and cultured in DMEM (Dulbecco's Modified Eagle Medium) containing 10% FBS (Fetal Bovine Serum) and 1% Penicillin-Streptomycin. Cells were incubated at 37° C. with 5% CO₂.

Transfection was performed on 12-well plates. Briefly, the Huh7 cells were grown overnight until the confluency was around 80%, then the mixture of 0.5 μg plasmid expressing FVIII-SQ or EGFP (Enhanced Green Fluorescent Protein) and 1.5 μL of PolyJet (SignaGen Laboratories, Maryland) was added to each well according to the manufacturer's protocol. The cells were transfected with each plasmid constructed above in duplicate. After 6-8 hours, cells were rinsed gently using DPBS (Dulbecco's Phosphate Buffered Saline) and cultured in F12 medium. The supernatants were collected 48 hours post-transfection to measure the activities and protein levels of FVIII-SQ by APTT and ELISA as discussed below.

Similarly, Huh7 cells were grown overnight on 12-well plates to perform transfection of plasmids with different promoters and luciferase coding sequences. The cells were transfected with each plasmid in triplicate. After 6-8 hours, Medium containing transfection reagents and plasmids were removed and fresh DMEM containing FBS and Penicillin-Streptomycin were added on 12-well plates. The cells were collected 24 h post transfection for measurement of luciferase activity.

### APTT (One Stage Activated Partial Thromboplastin Clotting Time)

For in vitro assay, ReFacto (Genetics Institute, Cambridge, MA) was serially diluted with F12 medium from 1 U/ml (200 ng/ml) by ½ till 1/64 dilutions and used as the standards. ReFacto is a recombinant FVIII and can be used as the standard for APTT and ELISA. The culture F12 medium was collected from the above mentioned 12-well plates and centrifuged at 13000 rpm for 5 minutes. The supernatants were then used as samples. 50 μL of STA-PTT reagent (Diagnostica Stago, Asnieres, France) was added to enough strips of STAGO cuvettes containing a magnetic bead in each well. Thereafter, each diluted standard protein and each sample was added to different wells of STAGO cuvettes all pre-filled with STA-PTT reagent. The mixtures were incubated at 37° C. for 170 seconds. Coagulation time was then initiated and measured by adding 50 μL of 25 mM CaCl₂) using STAGO machine (Diagnostica Stago, Asnieres, France). The FVIII-SQ activities were calculated according to the standard curve.

For in vivo assay, plasma was collected from posterior orbital venous plexus of mice by adding the blood samples to 1.5 mL tubes prefilled anticoagulant sodium citrate (final concentration 3.8%). Then supernatants, plasma of mice, were transferred into new tubes following centrifugation for 15 min at 2500 g. Plasma samples diluted in appropriate proportions were utilized for measurement of the activities and protein levels of FVIII-SQ by APTT and ELISA.

### ELISA (Enzyme-Linked Immunosorbent Assay)

In a 96-well plate, the wells were coated with 100 μl of the 2.5 ng/u L capture antibody PAH-FVIII-S (Haematologic Technologies, Essex) in coating buffer (containing 0.1 M sodium bicarbonate and carbonate, pH 9.6) for each well at 4° C. overnight. After the plates were washed 3 times for 5 minutes every time with 300 μL of PBST buffer (140 mM NaCl, 2.5 mM KCl, 8 mM Na₂HPO₄, 2 mM KH₂PO₄ and 0.05% Tween-20, pH 8.4), the wells were blocked with 300 μL of PBST buffer containing 3% BSA at room temperature for 2 hours. The wells are washed with PBST buffer three times, then 100 μL of the standards or samples were added and incubated at room temperature for 1.5 hours. Serial dilutions of ReFacto (12.5 ng/mL two-fold serially diluted to 0.1953 ng/ml) were used as the standards. After the wells were washed three times with PBST buffer, 100 μl of the 0.5 ng/μL detecting antibody biotin-labeled GMA-8021 (Green Mountain Antibodies, Burlington) was added. The plate was incubated at room temperature for 1 hour. After 3 times of washing, each well was added 100 μL 200-fold diluted Streptavidin-HRP (CST, Boston) in PBST buffer containing 0.1% BSA and incubated for 1 hour in the dark. Then the plate was washed three times with PBST buffer and the color was developed using 100 μL of KPL SureBlue TMB 1-Component Microwell Peroxidase Substrate (Seracare, Milford). The color development was carried out at room temperature for 1-10 min in the dark and stopped by adding 100 μL of 0.5 M H₂SO₄. The OD values were determined by a spectrophotometer at 450 nm and 630 nm. FVIII-SQ amounts in the medium were calculated according to the standard curve.

### Hydrodynamic Injection

Around 8-10 weeks old factor VIII deficient mice were injected with different rAAV vector plasmids via hydrodynamic injection. Briefly, each mouse was injected 2 mL mix containing PBS and 100 μg plasmid via tail vein gently at a constant speed. Three mice were treated for each group. Plasma of mice were collected 48 h post injection for measurement of the activities and protein levels of FVIII-SQ by APTT and ELISA.

### Luciferase Assays

The transfected cells on 12-well plates were rinsed gently using DPBS after removal of DMEM, then DPBS was removed and 100 μL cell lysis buffer in Firefly Luciferase Reporter Gene Assay Kit (Beyotime, Shanghai) was added to each well. Samples were transferred into Eppendorf tubes after 5 min at room temperature. After centrifuging at 4° C. and 12,000 rpm for 2 min, the supernatants were transferred into new tubes. 30 μL of each sample was added 30 μL of substrate in Firefly Luciferase Receptor Gene Assay Kit on 96-well white assay plate (Corning, New York), and the mixture was used for detecting luciferase activities by a Synergy H1 Hybrid Multi-Mode Microplate Reader (BioTek, Winooski).

### Data Analysis

The schematic pictures were produced by Adobe Illustrator CS5. Data statistical analysis was calculated by GraphPad Prism 8.0.1. All data was reported as mean±SD. The significant differences in FVIII-SQ activities and protein levels between HLP promoter and other promoter groups (or between other two promoter groups) were calculated by a two-tailed Student's t-test. The significant differences in luciferase activities between two promoter groups were also calculated by a two-tailed Student's t-test.

### Results

### Selection of Liver-Specific Core Promoters

The cargo capacity of rAAV vectors is limited, and an expression cassette containing a regular promoter and the therapeutic FVIII-SQ for treating Hemophilia A (HA) is too large to fit it. So small size promoters that could drive FVIII-SQ overexpression effectively are in urgent need. As it is known that core promoters are usually not specific enough or too weak for driving transgene overexpression. Here, a short, liver-specific core promoter was obtained first, which maintains the basal activity for transgene overexpression. Then the short liver-specific core promoter was combined with various small enhancers to construct synthetic promoters that drive strong gene expression.

To obtain small, effective and liver-specific promoter, the 219 bp hAAT promoter (hAAT, SEQ ID NO: 1) and the 94 bp hAATs promoter (hAATs, SEQ ID NO: 2) of human SERPINA1 genome (encoding human α-1 antitrypsin, hAAT) were selected. These two promoters were cloned into pssAAV-TTR-FVIII-SQ rAAV vector plasmid to obtain pssAAV-hAAT-FVIII-SQ and pssAAV-hAATs-FVIII-SQ, respectively. The hybrid liver-specific HLP promoter, 252 bp in length, containing regulatory elements from HCR (hepatic control region) and the sequence of human SERPINA1 genome, was selected as a positive control promoter based on the reference (Blood. 2013 Apr. 25; 121 (17): 3335-3344.) and the patent EP2698163A1. To check whether the activity of hAATs promoter can be enhanced by enhancers, pssAAV-HEx-hAATs-FVIII-SQ was constructed, which contains HEx-hAATs promoter (SEQ ID NO: 11), where HEx is a hybrid enhancer that is 66 bp in length derived from the HLP promoter (See FIG. 1, the panel showing HLP).

To check the activities of hAAT, hAATs and HEx-hAATs, different rAAV vector plasmids were made to place FVIII-SQ gene under the control of hAAT, hAATs, HEx-hAATs, and HLP, respectively. These vectors were then transfected in Huh7 cells on 12-well plates. Supernatants of cells were collected 48 hours post-transfection to measure the activities and protein levels of FVIII-SQ using APTT and ELISA. The data is shown in FIG. 4.

As seen in FIG. 4, the activity of FVIII-SQ under the control of hAAT promoter was decreased (P=0.0392) compared to that under the control of the liver-specific HLP promoter. However, the protein levels of FVIII-SQ under the control of the hAAT promoter were comparable (P=0.3572) to those under the control of the liver-specific HLP promoter in Huh7 cells. On the other hand, the activities (P=0.0009) and protein levels (P=0.0034) of FVIII-SQ under the control of the hAATs promoter were much lower than those under the control of the liver-specific HLP promoter in Huh7 cells. Nonetheless, the data shows that the hAATs promoter has maintained a basal activity to drive the expression of FVIII-SQ. Interestingly, as shown in FIG. 4, the activity of the hAATs promoter was restored by adding HEx, suggesting the hAATs promoter could be used to examine the effect of the synthetic enhancer.

Effect of Synthetic Enhancers on the hAATs Promoter

Several transcription factors, including HNF-1α/β, HNF-3β, DBP, HNF-4α, C/EBP-α/β, were reported to be transcription activators for controlling the expression of many liver-specific genes, such as albumin (ALB), transthyretin (TTR) and α-1 antitrypsin (AAT) genes. To explore whether a synthetic enhancer formed by randomly combining transcription factors binding sites (TFBS) of HNF-1α/β, HNF-3β, DBP, HNF-4α, C/EBP-α/β could enhance the activity of the hAATs promoter, a 54 bp enhancer Es was designed and synthesized (Es, SEQ ID NO: 3), which includes, from 5′ to 3′, the DNA binding sites of HNF-4α, HNF-3β, DBP, C/EBP-α/β and HNF-1α/β (See FIG. 2, top panel). Another 111 bp enhancer E2 was designed by adding a 52 bp enhancer Es-2 (similar to Es but with one thymine and one adenine deletion at the 5′ end and 3′ end of HNF-3β transcription factor binding site, respectively) behind Es (See FIG. 2, middle panel).

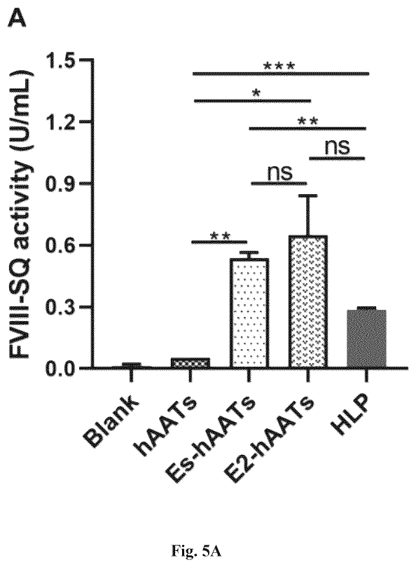

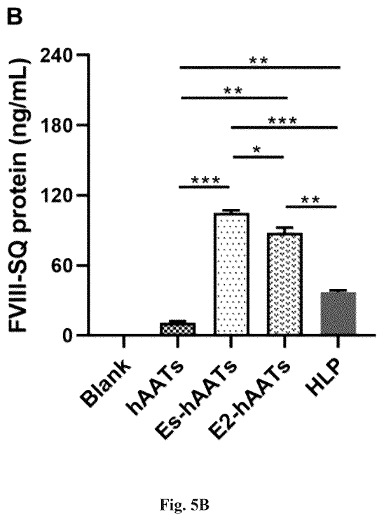

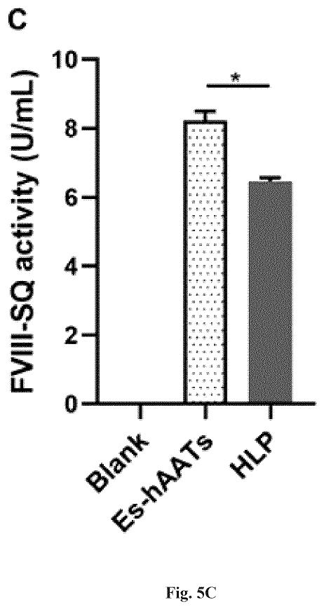

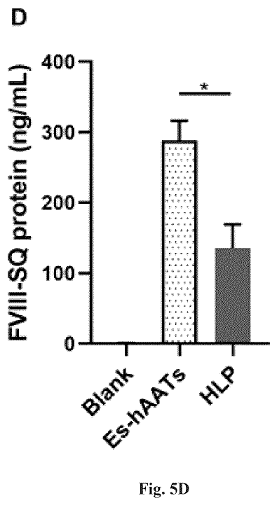

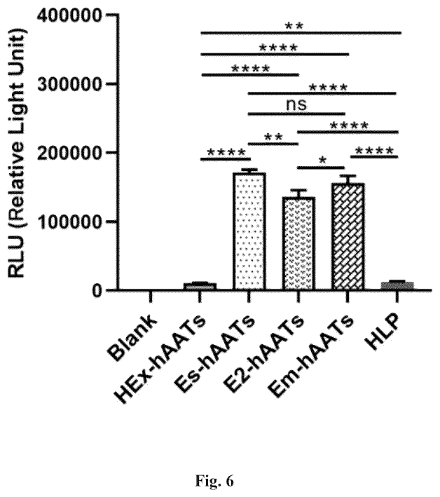

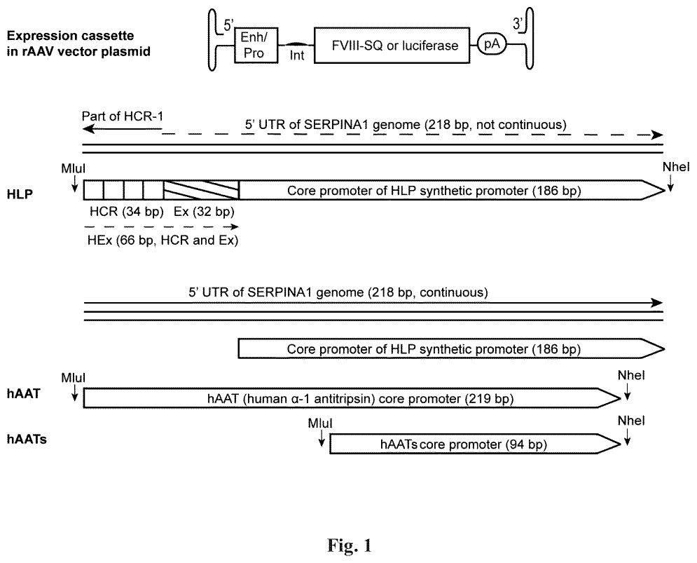

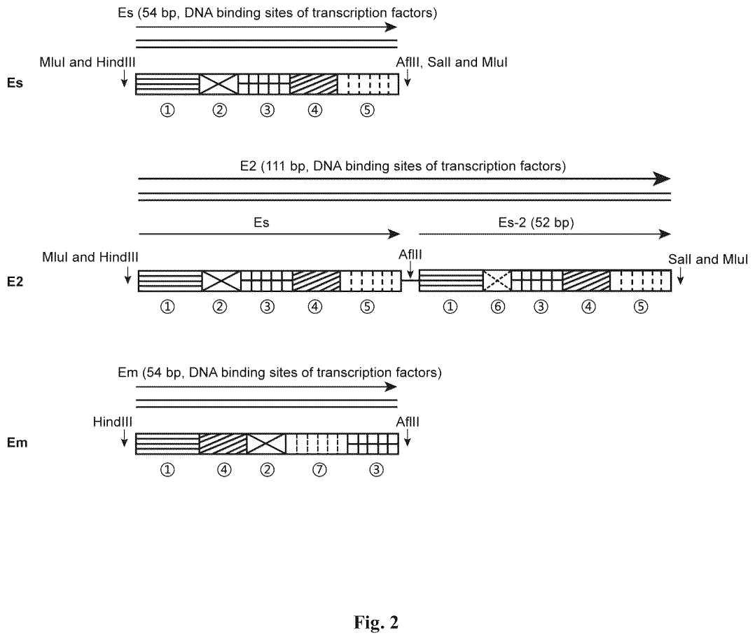

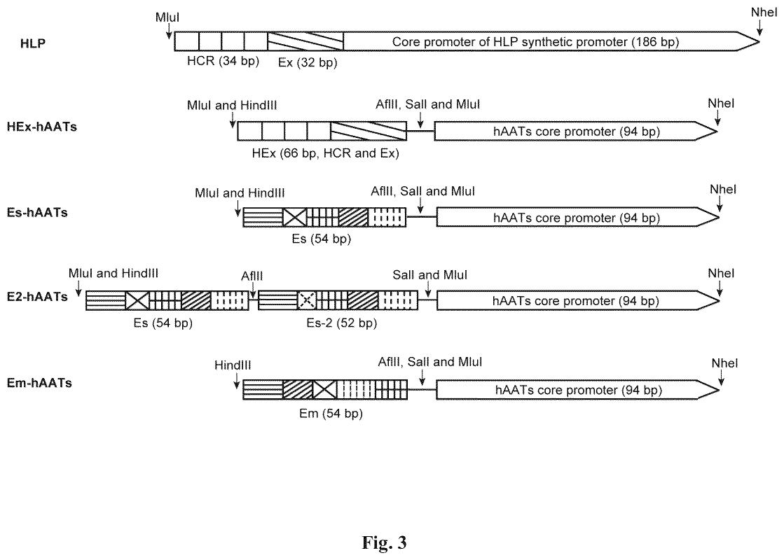

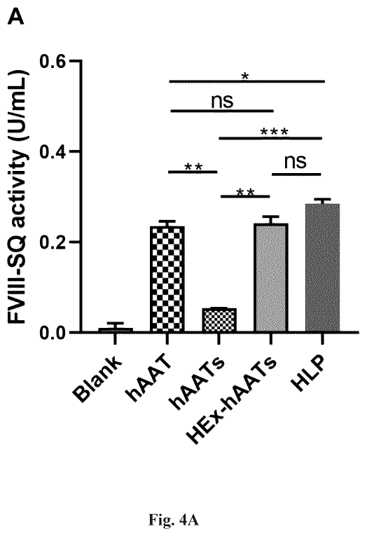

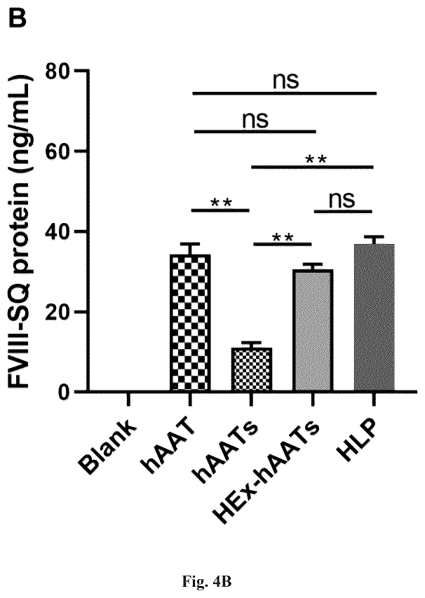

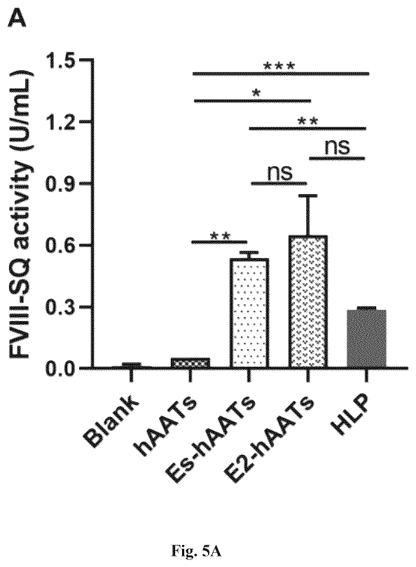

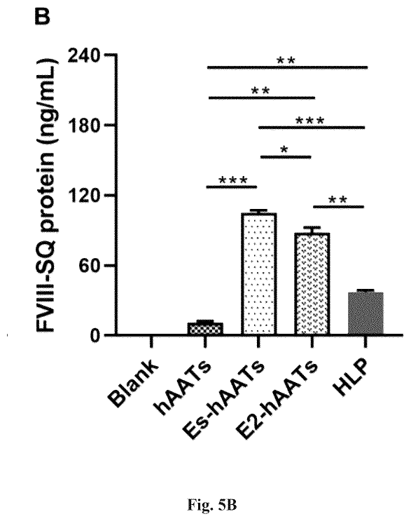

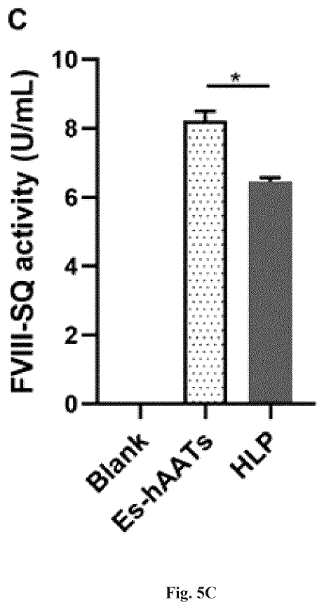

Es-hAATs synthetic promoter and E2-hAATs synthetic promoter are then created and their sequences are shown in SEQ ID NO: 10 and 13, respectively. Different rAAV vector plasmids were made to place FVIII-SQ gene under the control of Es-hAATs promoter, E2-hAATs promoter, or HLP promoter. Different rAAV vector plasmids were transfected in Huh7 cells on 12-well plates. Supernatants of cells were collected 48 hours post-transfection to measure the activities and protein levels of FVIII-SQ by APTT and ELISA. The data is shown in FIG. 5A and FIG. 5B.

As shown in FIG. 5A, the activities of FVIII-SQ driven by Es-hAATs promoter is significantly higher than that driven by the hAATs promoter (P=0.0016). Similarly, the protein levels of FVIII-SQ driven by Es-hAATs promoter is also significantly higher than that driven by the hAATs promoter (P=0.0004, FIG. 5B). These data suggests that the enhancer Es could remarkably enhance the activity of the hAATs promoter. Moreover, under the control of the Es-hAATs synthetic promoter (SEQ ID NO: 10), FVIII-SQ activity (P=0.0067) and protein levels (P=0.0009) were both around 2 times higher than those under the control of the HLP promoter, illustrating the strong gene expression enhancing ability of the enhancer Es.

As seen in FIG. 5A and FIG. 5B, both the activities and protein levels of FVIII-SQ driven by E2-hAATs promoter are significantly higher than those driven by the hAATs promoter. Comparing the activities and protein levels of FVIII-SQ, the E2-hAATs promoter also showed stronger promoting activity than HLP promoter. However, the data shows that there was no significant difference between the activities of FVIII-SQ driven by the Es-hAATs promoter and the E2-hAATs promoter, and the protein levels of FVIII-SQ driven by E2-hAATs promoter is significantly lower than that driven by the Es-hAATs promoter (P=0.0382, FIG. 5B). This observation is surprising. As seen in the middle panel of FIG. 2, the E2 enhancer contains more transcription factor binding sites (TFBS) than the Es enhancer. The data in FIG. 5A suggests that the addition of more TFBS did not enhance the promoting activity of the E2 enhancer as compared to the Es enhancer. Indeed, a negative effect was observed on protein level of FVIII-SQ using E2-hAATs promoter compared with Es-hAATs promoter (FIG. 5B).

Besides, in vivo verification was performed in factor VIII deficient mice. The rAAV vector plasmids where the FVIII-SQ gene is under the control of Es-hAATs promoter or HLP promoter have been injected into 8-10 weeks old FVIII deficient mice. Plasma of mice were collected 48 hours post injection for measurement of the activities and protein levels of FVIII-SQ by using APTT and ELISA. The data is shown in FIG. 5C and FIG. 5D.

As shown in FIG. 5C and FIG. 5D, the activities and protein levels of FVIII-SQ driven by Es-hAATs synthetic promoter in factor VIII deficient mice were both higher than the activity and protein level of FVIII-SQ driven by the HLP promoter (P=0.013 and P=0.0385). The result in mice was consistent with the results in Huh7 cells shown in FIG. 5A and FIG. 5B.

Comparison of the Activities of Different Enhancers on the hAATs Promoter

ITR in rAAV vector could act as a weak promoter (J Biol Chem. 1993 Feb. 15; 268 (5): 3781-90.), thus, ATG triple nucleotides in sequences of an enhancer or promoter might disrupt the translation of target proteins. Therefore, another 54 bp engineered enhancer (Em, SEQ ID NO: 9) was designed to remove ATG triple nucleotides by changing the combination of TFBS and replacing the seventh base guanine with cytosine in the sequences of TFBS of HNF-1α/β (See FIG. 2 bottom panel).

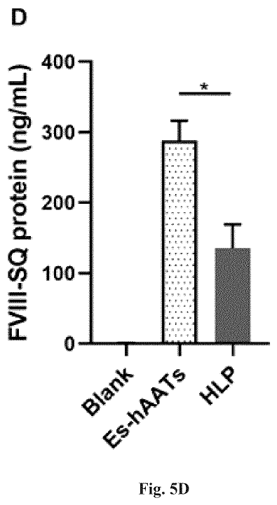

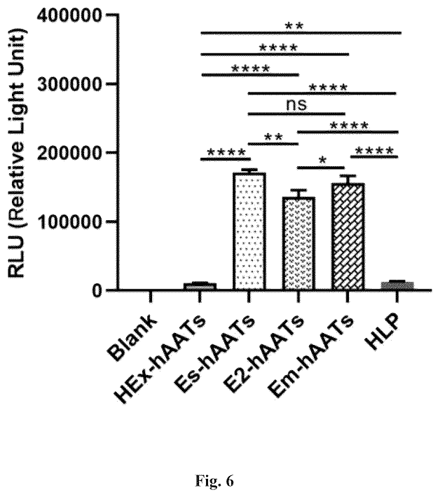

To examine the enhancing effects of different enhancers, synthetic promoters containing hAATs promoter and enhancer HEx, Es, E2 or Em were constructed (See FIG. 3). Different rAAV vector plasmids were made to place luciferase gene under the control of HEx-hAATs, Es-hAATs, E2-hAATs, Em-hAATs, and HLP, respectively. These vectors were then transfected in Huh7 cells on 12-well plates. Cells were collected 24 hours post-transfection to measure the activities of luciferase. The data is shown in FIG. 6.

As seen in FIG. 6, the luciferase activities driven by Es-hAATs and Em-hAATs were similar (P=0.0652), suggesting that the Em enhancer has similar enhancing activity as the Es enhancer for enhancing luciferase expression. Another observation is that the luciferase activities driven by the three synthetic promoters (Es-hAATs, E2-hAATs, and Em-hAATs) were remarkably stronger than the HEx-hAATs promoter and the HLP promoter (P<0.0001), suggesting that the Es, E2, and Em enhancers could remarkably enhance the promoting activity of the hAATs promoter.

### Example 2. Treatment of a Patient Suffering from a Genetic Disease

A human patient is tested to have a genetic disease.

An expression vector is made comprising the synthetic promoter disclosed herein operably linked to a therapeutic transgene. The effectiveness and safety of the expression vector are tested in in vitro cell culture and in vivo animal models before being used to treat the human patient.

### Example 3. Treatment of Human Hemophilia a Patient Using an rAAV Expression Vector

This example describes an exemplary method for the clinical use of rAAV vectors encoding FVIII-SQ for the treatment of hemophilia A.

An expression vector is made comprising the synthetic promoter disclosed herein operably linked to FVIII-SQ. The effectiveness and safety of the expression vector are tested in in vitro cell culture and in vivo animal models before being used to treat the human patient.

A patient diagnosed with hemophilia A is selected for treatment. The patient is administered a therapeutically effective amount of rAAV. The rAAV can be administered intravenously. An appropriate therapeutic dose can be selected by a medical practitioner.

In some cases, the therapeutically effective dose is in the range of 1×10¹¹ to 1×10¹⁴ viral particles (vp)/kg, such as about 1×10¹¹ vp/kg. In most instances, the patient is administered a single dose. The health of the subject can be monitored over time to determine the effectiveness of the treatment.

1. An engineered enhancer comprising one or more DNA binding sites for transcription factors, wherein each transcription factor is selected from the group consisting of HNF-4α, HNF-3β, D site-binding protein (DBP), CCAAT enhancer binding protein alpha/beta (C/EBP-α/β), and hepatocyte nuclear factor 1 alpha/beta (HNF-1α/β).

2. The engineered enhancer of claim 1, wherein the DNA binding sites for HNF-4α, HNF-3β, DBP, C/EBP-α/β and HNF-1α/β comprise nucleic acid sequences of SEQ ID NO: 4, SEQ ID NO: 5, SEQ ID NO: 6, SEQ ID NO: 7, and SEQ ID NO: 8, respectively.

3. The engineered enhancer of claim 1, wherein the engineered enhancer comprises a nucleic acid sequence having at least 70%, 75%, 80%, 85%, 90%, 95%, 99%, or 100% sequence identity to SEQ ID NO: 3, SEQ ID NO: 9, or SEQ ID NO: 12.

4. A synthetic promoter comprising an engineered enhancer of claim 1 and a nucleic acid sequence of a core promoter.

5. The synthetic promoter of claim 4, wherein the core promoter is a liver-specific promoter.

6. The synthetic promoter of claim 5, wherein the liver-specific promoter is a human α-1 antitrypsin (hAAT) promoter.

7. The synthetic promoter of claim 6, wherein the human α-1 antitrypsin (hAAT) promoter comprises a nucleic acid sequence having at least 80%, 85%, 90%, 95%, 99%, or 100% sequence identity to SEQ ID NO: 1 or SEQ ID NO: 2.

8. The synthetic promoter of claim 4, wherein the synthetic promoter comprises a nucleic acid sequence having at least 70%, 75%, 80%, 85%, 90%, 95%, 99%, or 100% sequence identity to SEQ ID NO: 10, SEQ ID NO: 13, or SEQ ID NO: 14.

9. An expression vector comprising a synthetic promoter of claim 4.

10. The expression vector of claim 9, further comprising a transgene operably linked to a synthetic promoter of claim 4.

11. The expression vector of claim 10, wherein the transgene encodes a therapeutic protein for treatment of a genetic disease or condition associated with liver.

12. The expression vector of claim 11, wherein the therapeutic protein is a Factor VIII protein or a functional fragment thereof.

13. The expression vector of claim 9, wherein the expression vector is a plasmid, a recombinant retroviral vector, a recombinant lentiviral vector, a recombinant adenoviral vector, or a recombinant adeno-associated viral vector (rAAV).

14. The expression vector of claim 13, wherein the expression vector is an rAAV vector.

15. A pharmaceutical composition comprising an enhancer of claim 1, a synthetic promoter of claim 4, or an expression vector of claim 9, and a pharmaceutically acceptable carrier.

16. A method of treating a genetic disease or condition associated with liver in a subject in need thereof, comprising administering a therapeutically effective amount of a pharmaceutical composition of claim 15 to the subject.

17. The method of claim 16, wherein the subject is a mammal.

18. (canceled)

19. The method of claim 16, wherein the genetic disease or condition associated with liver is selected from the group consisting of genetic cholestasis, hemophilia A, hemophilia B, phenylketonuria, hereditary hemochromatosis, tyrosinemia type 1, alpha-1 antitrypsin deficiency, argininosuccinic aciduria, liver cancer, glycogen storage disease, urea cycle disorder, Crigler-Najjar syndrome, familial amyloid polyneuropathy, atypical hemolytic uremic syndrome-1, primary hyperoxaluria type 1, maple syrup urine disease, acute intermittent porphyria, coagulation defects, GSD type1A, homozygous familial hypercholesterolemia, organic acidurias, cystic fibrosis, erythropoietic protoporphyria, Gaucher disease, familial hypercholesterolemia, ornithine and transcarbamylase deficiency.

20. (canceled)

21. (canceled)

22. A kit comprising a promoter of claim 4, or an expression vector of claim 9, or a pharmaceutical composition of claim 15.

23. The kit of claim 22, further comprising instructions for using contents of the kit.


SEQUENCE LISTING (27 sequences)

SEQ ID NO: 1 - DNA, 219 bp, Homo sapiens
  source 1..219: mol_type = other DNA
tgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagc

SEQ ID NO: 2 - DNA, 94 bp, Homo sapiens
  source 1..94: mol_type = other DNA
gtgaccttggttaatattcaccagcagcctcccccgttgcccctctggatccactgcttaaatacggacgaggacagggccctgtctcctcagc

SEQ ID NO: 3 - DNA, 54 bp, Synthetic construct
  source 1..54: mol_type = other DNA
tggactttgcacttgtaaacaattacgtaacattgcacaatgttaatgattaac

SEQ ID NO: 4 - DNA, 13 bp, Homo sapiens
  source 1..13: mol_type = other DNA
tggactttgcact

SEQ ID NO: 5 - skipped

SEQ ID NO: 6 - DNA, 10 bp, Homo sapiens
  source 1..10: mol_type = other DNA
attacgtaac

SEQ ID NO: 7 - DNA, 10 bp, Homo sapiens
  source 1..10: mol_type = other DNA
attgcacaat

SEQ ID NO: 8 - DNA, 13 bp, Homo sapiens
  source 1..13: mol_type = other DNA
gttaatgattaac

SEQ ID NO: 9 - DNA, 54 bp, Synthetic construct
  source 1..54: mol_type = other DNA
tggactttgcactattgcacaattgtaaacagttaatcattaacattacgtaac

SEQ ID NO: 10 - DNA, 164 bp, Synthetic construct
  source 1..164: mol_type = other DNA
tggactttgcacttgtaaacaattacgtaacattgcacaatgttaatgattaacttaagcgtcgacacgcgtgaccttggttaatattcaccagcagcctcccccgttgcccctctggatccactgcttaaatacggacgaggacagggccctgtctcctcagc

SEQ ID NO: 11 - DNA, 177 bp, Synthetic construct
  source 1..177: mol_type = other DNA
tgtttgctgcttgcaatgtttgcccattttagggtggacacaggacgctgtggtttctgagccaggcttaagcgtcgacacgcgtgaccttggttaatattcaccagcagcctcccccgttgcccctctggatccactgcttaaatacggacgaggacagggccctgtctcctcagc

SEQ ID NO: 12 - DNA, 52 bp, Synthetic construct
  source 1..52: mol_type = other DNA
tggactttgcactgtaaacattacgtaacattgcacaatgttaatgattaac

SEQ ID NO: 13 - DNA, 215 bp, Synthetic construct
  source 1..215: mol_type = other DNA
tggactttgcacttgtaaacaattacgtaacattgcacaatgttaatgattaacttaagtggactttgcactgtaaacattacgtaacattgcacaatgttaatgattaacgtcgacacgcgtgaccttggttaatattcaccagcagcctcccccgttgcccctctggatccactgcttaaatacggacgaggacagggccctgtctcctcagc

SEQ ID NO: 14 - DNA, 164 bp, Synthetic construct
  source 1..164: mol_type = other DNA
tggactttgcactattgcacaattgtaaacagttaatcattaacattacgtaacttaagcgtcgacacgcgtgaccttggttaatattcaccagcagcctcccccgttgcccctctggatccactgcttaaatacggacgaggacagggccctgtctcctcagc

SEQ ID NO: 15 - DNA, 32 bp, Synthetic construct
  source 1..32: mol_type = other DNA
accacgcgtgtttgctgcttgcaatgtttgcc

SEQ ID NO: 16 - DNA, 29 bp, Synthetic construct
  source 1..29: mol_type = other DNA
accgctagcgattcactgtcccaggtcag

SEQ ID NO: 17 - DNA, 30 bp, Synthetic construct
  source 1..30: mol_type = other DNA
accacgcgtgactcctttcggtaagtgcag

SEQ ID NO: 18 - DNA, 30 bp, Synthetic construct
  source 1..30: mol_type = other DNA
accgctagctgaggagacagggccctgtcc

SEQ ID NO: 19 - DNA, 29 bp, Synthetic construct
  source 1..29: mol_type = other DNA
accacgcgtgaccttggttaatattcacc

SEQ ID NO: 20 - DNA, 71 bp, Synthetic construct
  source 1..71: mol_type = other DNA
agcttgtttgctgcttgcaatgtttgcccattttagggtggacacaggacgctgtggtttctgagccaggc

SEQ ID NO: 21 - DNA, 71 bp, Synthetic construct
  source 1..71: mol_type = other DNA
ttaagcctggctcagaaaccacagcgtcctgtgtccaccctaaaatgggcaaacattgcaagcagcaaaca

SEQ ID NO: 22 - DNA, 77 bp, Synthetic construct
  source 1..77: mol_type = other DNA
cgcgtaagcttggactttgcacttgtaaacaattacgtaacattgcacaatgttaatgattaacttaagcgtcgaca

SEQ ID NO: 23 - DNA, 77 bp, Synthetic construct
  source 1..77: mol_type = other DNA
cgcgtgtcgacgcttaagttaatcattaacattgtgcaatgttacgtaattgtttacaagtgcaaagtccaagctta

SEQ ID NO: 24 - DNA, 58 bp, Synthetic construct
  source 1..58: mol_type = other DNA
ttaagtggactttgcactgtaaacattacgtaacattgcacaatgttaatgattaacg

SEQ ID NO: 25 - DNA, 58 bp, Synthetic construct
  source 1..58: mol_type = other DNA
tcgacgttaatcattaacattgtgcaatgttacgtaatgtttacagtgcaaagtccac

SEQ ID NO: 26 - DNA, 58 bp, Synthetic construct
  source 1..58: mol_type = other DNA
agcttggactttgcactattgcacaattgtaaacagttaatcattaacattacgtaac

SEQ ID NO: 27 - DNA, 58 bp, Synthetic construct
  source 1..58: mol_type = other DNA
ttaagttacgtaatgttaatgattaactgtttacaattgtgcaatagtgcaaagtcca